(19) United States
(12) Patent Application Publication
(10) Pub. No.: US 2023/0248585 A1
(43) Pub. Date: Aug. 10, 2023

(54) REUSABLE OUTER COVER FORMED FROM A NONWOVEN

(71) Applicant: Dow Global Technologies LLC, Midland, MI (US)

(72) Inventors: Brenda Colegrove, Lake Jackson, TX (US); Yinglong Chen, Lake Jackson, TX (US); William C. Glover, Pearland, TX (US); David M. Pearson, Lake Jackson, TX (US); Stacy L. Pesek, Pearland, TX (US); Jeremy Chris Reyes, Lake Jackson, TX (US)

(21) Appl. No.: 18/004,818

(22) PCT Filed: Jul. 6, 2021
(86) PCT No.: PCT/US2021/040505

§ 371 Date: Jan. 9, 2023

Related U.S. Application Data
(60) Provisional application No. 63/052,774, filed on Jul. 16, 2020.

(51) Int. Cl. A61F 13/49; A61F 13/505; D04H 1/4291; D04H 1/541

(52) U.S. Cl. CPC A61F 13/49003; A61F 13/4906; A61F 13/505; D04H 1/4291; D04H 1/5412

(57) ABSTRACT

Provided is a reusable outer cover. The reusable outer cover is suitable for use as a cover to an absorbent article. The reusable outer cover according to embodiments disclosed herein can be a single layer. It can be fully compatible with polyethylene recycling streams and can exhibit improved, maintained, or desirable properties in comparison to existing commercially available reusable outer covers. The reusable outer cover is formed from a nonwoven, where the nonwoven comprises a bicomponent fiber comprising a first and second region.

### TECHNICAL FIELD

Embodiments of the present disclosure generally relate to a reusable outer cover formed from a nonwoven comprising a bicomponent fiber.

### INTRODUCTION

Wearable absorbent articles, such as diapers, adult incontinence, and feminine hygiene products, include different layers. For example, a conventional diaper may consist of a topsheet formed from a polypropylene nonwoven, a backsheet formed from a polyethylene film, an acquisition distribution layer formed from a polyester nonwoven, and an absorbent core including equal amounts of superabsorbent polymer material and cellulose fluff pulp. Wearable articles are often not reusable (i.e., the articles and layers are disposed of after a single use), which results in waste. Waste can accumulate even more when articles are not recyclable. To combat waste, environmental sustainability initiatives are pushing industry towards creating articles that can be used again or multiple times (i.e., are reusable) and are recyclable at end-of-life. For example, a conventional diaper can be disposed of after a single use, but an outer cover can replace a backsheet or other layers of a conventional diaper. When the outer cover is combined with an absorbent article (e.g., an absorbent layer, insert, core, or nonwoven), it can provide a reusable and sustainable way of absorbing material. Problems exist, however, with creating a reusable outer cover for a wearable absorbent article that is comfortable, breathable, soft, durable, and/or recyclable.

### SUMMARY

Disclosed herein is a reusable outer cover. The reusable outer cover is suitable for use as a cover to an absorbent article. In embodiments, the reusable outer cover has a front region, a back region, and a crotch region disposed longitudinally between the front region and the back region, and a wearer-facing surface disposed opposite a garment-facing surface, and the reusable outer cover is formed from a nonwoven comprising a bicomponent fiber comprising a first region and a second region; the first region comprising a first polymer; and the second region comprising a second polymer.

These and other embodiments are described in more detail in the Detailed Description.

### BRIEF DESCRIPTION OF THE DRAWINGS

FIG. 1 is a schematic of a single reactor data flow diagram.

FIG. 2 is a schematic of a dual reactor data flow diagram.

FIG. 3 is an illustration and example of a reusable outer cover according to embodiments disclosed herein.

### DETAILED DESCRIPTION

Aspects of the disclosed reusable outer cover are described in more detail below. The reusable outer cover is suitable for use as a diaper cover, and can be used for a wide variety of applications, including, for example, as an outer cover for adult incontinence, feminine hygiene products, and other wearable absorbent articles. Accordingly, the reusable outer cover as a diaper cover is merely an illustrative implementation of the embodiments disclosed herein. The embodiments are applicable to other technologies that are susceptible to similar problems as those discussed above.

As used herein, the terms “comprising,” “including,” “having,” and their derivatives, are not intended to exclude the presence of any additional component, step or procedure, whether or not the same is specifically disclosed. In order to avoid any doubt, all compositions claimed through use of the term “comprising” may include any additional additive, adjuvant, or compound, whether polymeric or otherwise, unless stated to the contrary. In contrast, the term, “consisting essentially of” excludes from the scope of any succeeding recitation any other component, step or procedure, excepting those that are not essential to operability. The term “consisting of” excludes any component, step or procedure not specifically delineated or listed.

As used herein, the term “interpolymer” refers to polymers prepared by the polymerization of at least two different types of monomers. The term interpolymer thus includes copolymers (employed to refer to polymers prepared from two different types of monomers), and polymers prepared from more than two different types of monomers.

As used herein, the term “polymer” means a polymeric compound prepared by polymerizing monomers, whether of the same or a different type. The term polymer thus encompasses the term homopolymer (employed to refer to polymers prepared from only one type of monomer, with the understanding that trace amounts of impurities can be incorporated into the polymer structure), and the term interpolymer as defined above. A polymer may be a single polymer or polymer blend.

As used herein, the term “polyethylene” refers to polymers comprising greater than 50% by weight of units which are derived from ethylene monomer, and optionally, one or more comonomers. This may include polyethylene homopolymers or copolymers (meaning units derived from two or more comonomers). Common forms of polyethylene known in the art include Low Density Polyethylene (LDPE); Linear Low Density Polyethylene (LLDPE); Ultra Low Density Polyethylene (ULDPE); Very Low Density Polyethylene (VLDPE); single-site catalyzed Linear Low Density Polyethylene, including both linear and substantially linear low density resins (m-LLDPE); Medium Density Polyethylene (MDPE); and High Density Polyethylene (HDPE).

As used herein, the term “polypropylene” refers to polymers comprising greater than 50%, by weight, of units derived from propylene monomer, and optionally, one or more comonomers. This may include homopolymer polypropylene, random copolymer polypropylene, impact copolymer polypropylene, and propylene-based plastomers or elastomers (“PBE” or “PBPE”). PBE or PBPE polymers are further described in detail in the U.S. Pat. Nos. 6,960,635 and 6,525,157, incorporated herein by reference. Such polymers are commercially available from The Dow Chemical Company, under the tradename VERSIFY™ or from ExxonMobil Chemical Company, under the tradename VISTAMAXX™.

As used herein, the term “polyethylene terephthalate” (PET) refers to a polyester formed by the condensation of ethylene glycol and terephthalic acid.

As used herein, the terms “nonwoven,” “nonwoven web,” and “nonwoven fabric” are used herein interchangeably. “Nonwoven” refers to a web or fabric having a structure of individual fibers or threads which are randomly interlaid, but not in an identifiable manner as is the case for a knitted fabric.

As used herein, the term “meltblown” refers to the fabrication of nonwoven fabrics via a process which generally includes the following steps: (a) extruding molten thermoplastic strands from a spinneret; (b) simultaneously quenching and attenuating the polymer stream immediately below the spinneret using streams of high velocity heated air; (c) collecting the drawn strands into a web on a collecting surface. Meltblown webs can be bonded by a variety of means including, but not limited to, autogeneous bonding, i.e., self bonding without further treatment, thermo-calendaring process, adhesive bonding process, hot air bonding process, needle punch process, hydroentangling process, and combinations thereof.

As used herein, the term “spunbond” refers to the fabrication of nonwoven fabric including the following steps: (a) extruding molten thermoplastic strands from a plurality of fine capillaries called a spinneret; (b) quenching the strands with a flow of air which is generally cooled in order to hasten the solidification of the molten strands; (c) attenuating the stands by advancing them through the quench zone with a draw tension that can be applied by either pneumatically entraining the stands in an air stream or by winding them around mechanical draw rolls of the type commonly used in the textile fibers industry; (d) collecting the drawn strands into a web on a foraminous surface, e.g., moving screen or porous belt; and (e) bonding the web of loose strands into a nonwoven fabric. Bonding can be achieved by a variety of means including, but not limited to, thermo-calendaring process, adhesive bonding process, hot air bonding process, needle punch process, hydroentangling process, and combinations thereof.

As used herein, the term “absorbent article” refers to material that absorbs and contains body exudates and that is placed against or in proximity to the body of a wearer to absorb and contain extrudates discharged from the body.

As used herein, the term “reusable” refers to a material capable of being restored or reused for more than one usage cycle (e.g., a diaper change).

As used herein, the term “launderable” refers to a material that is configured to withstand a multiple number (e.g., at least 5) of cycles of machine washing, without significant degradation in structure or function.

### Bicomponent Fibers of the Nonwoven of the Reusable Outer Cover

The bicomponent fiber according to embodiments of the present disclosure can be formed into a fiber via different techniques, for example, via melt spinning. In melt spinning, the first region and second region can be melted, coextruded, and forced through fine orifices in a metallic plate, spinneret, into air or other gas, where the coextruded regions are cooled and solidified for forming bicomponent fibers. The solidified filaments can be drawn off via air jets, rotating rolls, or godets, and can be laid on a conveyer belt as a web for forming a nonwoven. The bicomponent fiber according to embodiments of the present disclosure contains two regions (i.e., a first region and a second region). The bicomponent fiber according to embodiments of the present disclosure can be arranged in a variety of different configurations. Examples of bicomponent fiber configurations are core-sheath, side-by-side, segmented pie, or islands-in-the-sea. In some embodiments, the bicomponent fiber may have a core-sheath configuration wherein a cross section of the fiber shows one region, a core, surrounded by another region, a sheath. In other embodiments, the bicomponent fibers may have a side-by-side configuration. In further embodiments, the bicomponent fibers may have a segmented pie configuration wherein a cross section of the fiber shows one region occupying a portion, for example a quarter, a third, a half of the cross section and a second region occupies the remainder of the cross section. In even further embodiments, the bicomponent fiber may have an islands-in-the-sea configuration, where the first region is multiple core regions (also referred to as islands) and the second region is a sheath region (also referred to as a sea), and the sheath region or sea surrounds the multiple core regions or islands.

The bicomponent fiber disclosed herein has a first region and a second region. The regions of the bicomponent fibers relate to the compositions that are extruded from the spinneret. For example, in a core-sheath configuration, the first region can be the core and the second region can be the sheath.

In embodiments, the bicomponent fiber comprises a first region and a second region, wherein the weight ratio of the first region to the second region is 10:90 to 90:10. All individual values and subranges of a ratio of from 90:10 to 10:90 are disclosed and included herein. For example, in embodiments, the weight ratio of the first region to the second region can be from 80:20 to 20:80, from 70:30 to 30:70, from 60:40 to 40:60, or from 55:45 to 45:55.

In embodiments, the bicomponent fiber has a denier of less than 50 g/9000 m. All individual values and subranges of less than 50 g/9000 m are disclosed and included herein. For example, the bicomponent fiber can have a denier of less than 40, less than 30, less than 20, less than 10, less than 5, less than 3, less than 2, less than 1.5, or less than 1.2 g/9000 m, or can have a denier in the range of from 0.1 to 50, 0.1 to 40, 0.1 to 30, 0.1 to 20, 0.1 to 10, 0.1 to 5, 0.1 to 2.0, 0.1 to 1.5, 0.1 to 1.2, 1 to 50, 1 to 40, 1 to 30, 1 to 20, 1 to 10, 1 to 5, 1 to 2.0, 1 to 1.5, or 1 to 1.2 g/9000 m.

### First Region of Bicomponent Fiber

In embodiments, the first region comprises a first polymer in an amount of at least 75 wt. % based on total weight of the first region. In some embodiments, the first polymer can comprise from 75 to 100 wt. % of the total weight of the first region. All individual values and subranges of at least 75 wt. % are included herein and disclosed herein; for example, the first polymer can be from a lower limit of 75, 80, 85, 90, or 95 wt. % to an upper limit of 80, 85, 90, 95, 100 wt. % based on total weight of the first region.

The first polymer according to embodiments of the present disclosure is a polypropylene, a polyethylene, a polyethylene terephthalate, or a combination or blend thereof. In some embodiments, the first region may further comprise additional components, such as, one or more other polymers and/or one or more additives. Such additives include, but are not limited to, antistatic agents, color enhancers, dyes, lubricants, fillers, pigments, primary antioxidants, secondary antioxidants, processing aids, UV stabilizers, anti-blocks, slip agents, tackifiers, fire retardants, anti-microbial agents, odor reducer agents, anti-fungal agents, and combinations thereof. Effective amounts of additives are known in the art and depend on parameters of the polymers in the composition and conditions to which they are exposed.

In one embodiment, the first polymer is a first ethylene/alpha-olefin interpolymer. The term “ethylene/alpha-olefin interpolymer” refers to a polyethylene comprising ethylene and an alpha-olefin having 3 or more carbon atoms. In embodiments, the first ethylene/alpha-olefin interpolymer comprises greater than 55 wt. % of the units derived from ethylene and less than 45 wt. % of the units derived from one or more alpha-olefin comonomers (based on the total amount of polymerizable monomers). All individual values and subranges of greater than 55 wt. % of the units derived from ethylene and less than 45 wt. % of the units derived from one or more alpha-olefin comonomers are included and disclosed herein. For example, in embodiments, the first ethylene/alpha-olefin interpolymer can comprise (a) greater than 55%, greater than 70%, greater than 85%, greater than 90%, greater than 92%, greater than 95%, greater than 97%, greater than 98%, greater than 99%, greater than 99.5%, or from 55% to 99.5%, from 55% to 95% from 55% to 90%, from 55% to 99.5%, from 55% to 99%, from 55% to 97%, from 55% to 94%, from 75% to 90%, from 90% to 99.9%, from 90% to 99.5% from 90% to 97%, or from 90% to 95% by weight, of the units derived from ethylene; and (b) less than 45%, less than 30%, less than 20%, less than 18%, less than 15%, less than 12%, less than 10%, less than 8%, less than 5%, less than 4%, less than 3%, less than 2%, less than 1%, or from 0.1 to 45%, from 0.1 to 25%, from 0.1 to 10%, from 0.1 to 5%, from 0.5 to 20%, from 0.5 to 10%, from 0.5 to 5%, from 1 to 20%, from 1 to 10%, from 1 to 5%, from 5 to 20%, or from 5 to 10%, by weight, of units derived from one or more α-olefin comonomers. The comonomer content may be measured using any suitable technique, such as techniques based on nuclear magnetic resonance (“NMR”) spectroscopy, and, for example, by 13C NMR analysis as described in U.S. Pat. No. 7,498,282, which is incorporated herein by reference.

Suitable alpha-olefin comonomers typically have no more than 20 carbon atoms. The one or more alpha-olefins of the first ethylene/alpha-olefin interpolymer may be selected from the group consisting of C3-C20 acetylenically unsaturated monomers and C4-C18 diolefins. For example, the alpha-olefin comonomers may have 3 to 10 carbon atoms, or 3 to 8 carbon atoms. Exemplary alpha-olefin comonomers include, but are not limited to, propylene, 1-butene, 1-pentene, 1-hexene, 1-heptene, 1-octene, 1-nonene, 1-decene, and 4-methyl-1-pentene. The one or more alpha-olefin comonomers of the first ethylene/alpha-olefin interpolymer may, for example, be selected from the group consisting of propylene, 1-butene, 1-hexene, and 1-octene; or in the alternative, from the group consisting of 1-butene, 1-hexene and 1-octene, or in the alternative, from the group consisting of 1-hexene and 1-octene. In one or more embodiments, the first ethylene/alpha-olefin interpolymer may comprise greater than 0 wt. % and less than 45 wt. % of units derived from one or more of 1-octene, 1-hexene, or 1-butene comonomers.

In embodiments, the first ethylene/alpha-olefin interpolymer has a density in the range of from 0.940 to 0.965 g/cm³. All individual values and subranges of a density in the range of from 0.940 to 0.965 g/cm³ are disclosed and included herein. For example, the first ethylene/alpha-olefin interpolymer can have a density in the range of from 0.940 to 0.965 g/cm³, 0.940 to 0.960 g/cm³, 0.945 to 0.965 g/cm³, 0.945 to 0.960 g/cm³, 0.950 to 0.965 g/cm³, or 0.950 to 0.960 g/cm³, where density can be measured according to ASTM D792.

In embodiments, the first ethylene/alpha-olefin interpolymer has a melt index (I2), measured according to ASTM D1238, 190° C., 2.16 kg, in the range of from 10 to 60 g/10 minutes. All individual values and subranges of from 10 to 60 g/10 minutes are included and disclosed herein. For example, in some embodiments, the first ethylene/alpha-olefin interpolymer can have a melt index (I2) in the range of from 10 to 60 g/10 minutes, from 10 to 50 g/10 minutes, from 10 to 40 g/10 minutes, from 10 to 30 g/10 minutes, from 10 to 20 g/10 minutes, from 20 to 60 g/10 minutes, from 20 to 50 g/10 minutes, from 20 to 40 g/10 minutes, from 20 to 30 g/10 minutes, from 15 to 60 g/10 minutes, from 15 to 50 g/10 minutes, from 15 to 40 g/10 minutes, from 15 to 30 g/10 minutes, or from 15 to 20 g/10 minutes, where melt index (I2) can be measured according to ASTM D1238, 190° C., 2.16 kg.

In embodiments, the first ethylene/alpha-olefin interpolymer has a molecular weight distribution, expressed as the ratio of the weight average molecular weight to number average molecular weight (M_{w(GPC)}/M_{n(GPC)}), of from 1.5 to 5.0. All individual values and subranges of a molecular weight distribution (M_{w(GPC)}/M_{n(GPC)}) of from 1.5 to 5.0 are disclosed and included herein. For example, the first ethylene/alpha-olefin interpolymer can have a molecular weight distribution (M_{w(GPC)}/M_{n(GPC)}) of from 1.5 to 5.0, 1.5 to 4.0, 1.5 to 3.0, 1.5 to 2.5, 2.0 to 5.0, 2.0 to 4.0, 2.0 to 3.0, or 2.0 to 2.5, where molecular weight distribution can be expressed as the ratio of the weight average molecular weight to number average molecular weight (M_{w(GPC)}/M_{n(GPC)}) and can be measured in accordance with the conventional Gel Permeation Chromatography (GPC) test method described below.

In embodiments, the first ethylene/alpha-olefin interpolymer has a highest crystallization temperature (Tc) in the range of from 108° C. to 118° C., where highest crystallization temperature (Tc) can be measured according to the DSC test method described below. All individual values and subranges of from 108° C. to 118° C. are disclosed and included herein. For example, the first ethylene/alpha-olefin interpolymer can have a highest crystallization temperature (Tc) in the range of from 108° C. to 118° C., 110° C. to 118° C., 112° C. to 118° C., 108° C. to 116° C., 108° C. to 115° C., 110° C. to 116° C., 110° C. to 115° C., 112° C. to 116° C., or 113° C. to 115° C., when measured according to the DSC test method described below.

### Second Region of Bicomponent Fiber

In embodiments, the second region comprises a second polymer in an amount of at least 75 wt. % based on total weight of the second region. In some embodiments, the second polymer can comprise from 75 to 100 wt. % of the total weight of the second region. All individual values and subranges of at least 75 wt. % are included herein and disclosed herein; for example, the second polymer can be from a lower limit of 75, 80, 85, 90, or 95 wt. % to an upper limit of 80, 85, 90, 95, 100 wt. % based on total weight of the second region.

The second polymer according to embodiments of the present disclosure is a polypropylene, a polyethylene, a polyethylene terephthalate, or a combination or blend thereof. In some embodiments, the second region may further comprise additional components, such as, one or more other polymers and/or one or more additives. Such additives include, but are not limited to, antistatic agents, color enhancers, dyes, lubricants, fillers, pigments, primary antioxidants, secondary antioxidants, processing aids, UV stabilizers, anti-blocks, slip agents, tackifiers, fire retardants, anti-microbial agents, odor reducer agents, anti-fungal agents, and combinations thereof. Effective amounts of additives are known in the art and depend on parameters of the polymers in the composition and conditions to which they are exposed.

In one embodiment, the second polymer is a second ethylene/alpha-olefin interpolymer. In such embodiments, the second ethylene/alpha-olefin interpolymer comprises greater than 55 wt. % of the units derived from ethylene and less than 45 wt. % of the units derived from one or more alpha-olefin comonomers (based on the total amount of polymerizable monomers). All individual values and subranges of greater than 55 wt. % of the units derived from ethylene and less than 45 wt. % of the units derived from one or more alpha-olefin comonomers are included and disclosed herein. For example, the second ethylene/alpha-olefin interpolymer can comprise (a) greater than 55%, greater than 70%, greater than 85%, greater than 90%, greater than 92%, greater than 95%, greater than 97%, greater than 98%, greater than 99%, greater than 99.5%, or from 55% to 99.5%, from 55% to 95% from 55% to 90%, from 55% to 99.5%, from 55% to 99%, from 55% to 97%, from 55% to 94%, from 75% to 90%, from 90% to 99.9%, from 90% to 99.5% from 90% to 97%, or from 90% to 95% by weight, of the units derived from ethylene; and (b) less than 45%, less than 30%, less than 20%, less than 18%, less than 15%, less than 12%, less than 10%, less than 8%, less than 5%, less than 4%, less than 3%, less than 2%, less than 1%, or from 0.1 to 45%, from 0.1 to 25%, from 0.1 to 10%, from 0.1 to 5%, from 0.5 to 20%, from 0.5 to 10%, from 0.5 to 5%, from 1 to 20%, from 1 to 10%, from 1 to 5%, from 5 to 20%, or from 5 to 10%, by weight, of units derived from one or more α-olefin comonomers. The comonomer content may be measured using any suitable technique, such as techniques based on nuclear magnetic resonance (“NMR”) spectroscopy, and, for example, by 13C NMR analysis as described in U.S. Pat. No. 7,498,282, which is incorporated herein by reference.

Suitable alpha-olefin comonomers typically have no more than 20 carbon atoms. The one or more alpha-olefins of the second ethylene/alpha-olefin interpolymer may be selected from the group consisting of C3-C20 acetylenically unsaturated monomers and C4-C18 diolefins. For example, the alpha-olefin comonomers may have 3 to 10 carbon atoms, or 3 to 8 carbon atoms. Exemplary alpha-olefin comonomers include, but are not limited to, propylene, 1-butene, 1-pentene, 1-hexene, 1-heptene, 1-octene, 1-nonene, 1-decene, and 4-methyl-1-pentene. The one or more alpha-olefin comonomers of the second ethylene/alpha-olefin interpolymer may, for example, be selected from the group consisting of propylene, 1-butene, 1-hexene, and 1-octene; or in the alternative, from the group consisting of 1-butene, 1-hexene and 1-octene, or in the alternative, from the group consisting of 1-hexene and 1-octene. In one or more embodiments, the second ethylene/alpha-olefin interpolymer may comprise greater than 0 wt. % and less than 45 wt. % of units derived from one or more of 1-octene, 1-hexene, or 1-butene comonomers.

In embodiments, the second ethylene/alpha-olefin interpolymer has a density that is less than the density of the first ethylene/alpha-olefin interpolymer. In other embodiments, the second ethylene/alpha-olefin interpolymer and the first ethylene/alpha-olefin interpolymer are the same, and the second ethylene/alpha-olefin interpolymer has a density equal to the density of the first ethylene/alpha-olefin interpolymer.

In embodiments, the second ethylene/alpha-olefin interpolymer has a density in the range of from 0.880 to 0.965 g/cm³. All individual values and subranges of a density in the range of from 0.880 to 0.965 g/cm³ are disclosed and included herein. For example, in some embodiments, the second ethylene/alpha-olefin interpolymer can have a density in the range of from 0.880 to 0.965 g/cm³, 0.880 to 0.950 g/cm³, 0.880 to 0.935 g/cm³, 0.890 to 0.950 g/cm³, 0.890 to 0.930 g/cm³, 0.890 to 0.910 g/cm³, 0.890 to 0.900 g/cm³, 0.900 to 0.965 g/cm³, 0.900 to 0.950 g/cm³, 0.900 to 0.930 g/cm³, or 0.900 to 0.910 g/cm³, where density can be measured according to ASTM D792.

In embodiments, the second ethylene/alpha-olefin interpolymer has a melt index (I2), measured according to ASTM D1238, 190° C., 2.16 kg, in the range of from 10 to 60 g/10 minutes. All individual values and subranges of from 10 to 60 g/10 minutes are included and disclosed herein. For example, in some embodiments, the second ethylene/alpha-olefin interpolymer can have a melt index (I2) in the range of from 10 to 60 g/10 minutes, from 10 to 50 g/10 minutes, from 10 to 40 g/10 minutes, from 10 to 30 g/10 minutes, from 10 to 20 g/10 minutes, from 20 to 60 g/10 minutes, from 20 to 50 g/10 minutes, from 20 to 40 g/10 minutes, from 20 to 30 g/10 minutes, from 15 to 60 g/10 minutes, from 15 to 50 g/10 minutes, from 15 to 40 g/10 minutes, from 15 to 30 g/10 minutes, or from 15 to 20 g/10 minutes, where melt index (I2) can be measured according to ASTM D1238, 190° C., 2.16 kg.

In embodiments, the second ethylene/alpha-olefin interpolymer has a molecular weight distribution, expressed as the ratio of the weight average molecular weight to number average molecular weight (M_{w(GPC)}/M_{n(GPC)}), of from 1.5 to 5.0. All individual values and subranges of a molecular weight distribution (M_{w(GPC)}/M_{n(GPC)}) of from 1.5 to 5.0 are disclosed and included herein. For example, the second ethylene/alpha-olefin interpolymer can have a molecular weight distribution (M_{w(GPC)}/M_{n(GPC)}) of from 1.5 to 5.0, 1.5 to 4.0, 1.5 to 3.0, 1.5 to 2.5, 2.0 to 5.0, 2.0 to 4.0, 2.0 to 3.0, or 2.0 to 2.5, where molecular weight distribution can be expressed as the ratio of the weight average molecular weight to number average molecular weight (M_{w(GPC)}/M_{n(GPC)}) and can be measured in accordance with the conventional Gel Permeation Chromatography (GPC) test method described below.

In embodiments, the second ethylene/alpha-olefin interpolymer has a highest crystallization temperature (Tc) in the range of from 90° C. to 118° C., where highest crystallization temperature (Tc) can be measured according to the DSC test method described below. All individual values and subranges of from 90° C. to 118° C. are disclosed and included herein. For example, the second ethylene/alpha-olefin interpolymer can have a highest crystallization temperature (Tc) in the range of from 90° C. to 118° C., 90° C. to 110° C., 90° C. to 105° C., 95° C. to 100° C., 95° C. to 118° C., 95° C. to 110° C., or 95° C. to 100° C., where highest crystallization temperature (Tc) can be measured according to the DSC test method described below.

### Synthesis of First or Second Ethylene/Alpha-Olefin Interpolymers

Any conventional polymerization processes can be employed to produce the first or second ethylene/alpha-olefin interpolymer described above. Such conventional polymerization processes include, but are not limited to, solution polymerization process, using one or more conventional reactors e.g. loop reactors, isothermal reactors, stirred tank reactors, batch reactors in parallel, series, and/or any combinations thereof. Such conventional polymerization processes also include gas-phase, solution or slurry polymerization or any combination thereof, using any type of reactor or reactor configuration known in the art.

In embodiments, the solution phase polymerization process occurs in one or more well-stirred reactors such as one or more loop reactors at a temperature in the range of from 115 to 250° C.; for example, from 155 to 225° C., and at pressures in the range of from 300 to 1000 psi; for example, from 400 to 750 psi. In one embodiment in a dual reactor, the temperature in the first reactor temperature is in the range of from 115 to 190° C., for example, from 115 to 150° C., and the second reactor temperature is in the range of 150 to 200° C., for example, from 170 to 195° C. In another embodiment in a single reactor, the temperature in the reactor temperature is in the range of from 115 to 250° C., for example, from 155 to 225° C. The residence time in a solution phase polymerization process is typically in the range of from 2 to 30 minutes; for example, from 10 to 20 minutes. Ethylene, solvent, one or more catalyst systems, optionally one or more cocatalysts, optionally one or more impurity scavengers, and optionally one or more comonomers are fed continuously to one or more reactors. Exemplary solvents include, but are not limited to, isoparaffins. For example, such solvents are commercially available under the name ISOPAR E from ExxonMobil Chemical Co., Houston, Tex. The resultant mixture of the first or second polyethylene composition and solvent is then removed from the reactor and the first or second polyethylene composition is isolated. Solvent is typically recovered via a solvent recovery unit, i.e. heat exchangers and vapor liquid separator drum, and is then recycled back into the polymerization system.

In one embodiment, the first or second ethylene/alpha-olefin interpolymer may be produced via a solution polymerization process in a dual reactor system, for example a dual loop reactor system, wherein ethylene and optionally one or more a-olefins are polymerized in the presence of one or more catalyst systems. Additionally, one or more co-catalysts may be present. In another embodiment, the first or second ethylene/alpha-olefin interpolymer may be produced via a solution polymerization process in a single reactor system, for example a single loop reactor system, wherein ethylene and optionally one or more a-olefins are polymerized in the presence of one or more catalyst systems.

An example of a catalyst system suitable for producing the first or second ethylene/alpha-olefin interpolymer can be a catalyst system comprising a procatalyst component comprising a metal-ligand complex of formula (I):

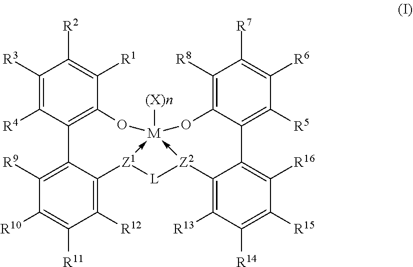

In formula (I), M is a metal chosen from titanium, zirconium, or hafnium, the metal being in a formal oxidation state of +2, +3, or +4; n is 0, 1, or 2; when n is 1, X is a monodentate ligand or a bidentate ligand; when n is 2, each X is a monodentate ligand and is the same or different; the metal-ligand complex is overall charge-neutral; each Z is independently chosen from —O—, —S—, —N(R^{N})—, or —P(R^{P})—, wherein independently each R^{N} and R^{P} is (C1-C30)hydrocarbyl or (C1-C30)heterohydrocarbyl; L is (C₁-C₄₀)hydrocarbylene or (C₁-C₄₀)heterohydrocarbylene, wherein the (C₁-C₄₀)hydrocarbylene has a portion that comprises a 1-carbon atom to 10-carbon atom linker backbone linking the two Z groups in Formula (I) (to which L is bonded) or the (C₁-C₄₀)heterohydrocarbylene has a portion that comprises a 1-atom to 10-atom linker backbone linking the two Z groups in Formula (I), wherein each of the 1 to 10 atoms of the 1-atom to 10-atom linker backbone of the (C₁-C₄₀)heterohydrocarbylene independently is a carbon atom or heteroatom, wherein each heteroatom independently is O, S, S(O), S(O)₂, Si(R^{C})₂, Ge(R^{C})₂, P(R^{C}), or N(R^{C}), wherein independently each R^{C} is (C₁-C₃₀)hydrocarbyl or (C₁-C₃₀)heterohydrocarbyl; R¹ and R⁸ are independently selected from the group consisting of —H, (C₁-C₄₀)hydrocarbyl, (C₁-C₄₀)heterohydrocarbyl, —Si(R^{C})₃, —Ge(R^{C})₃, —P(R^{P})₂, —N(R^{N})₂, —OR^{C}, —SR^{C}, —NO₂, —CN, —CF₃, R^{C}S(O)—, R^{C}S(O)₂—, (R^{C})₂C═N—, R^{C}C(O)O—, R^{C}OC(O)—, R^{C}C(O)N(R^{N})—, (R^{N})₂NC(O)—, halogen, and radicals having formula (II), formula (III), or formula (IV):

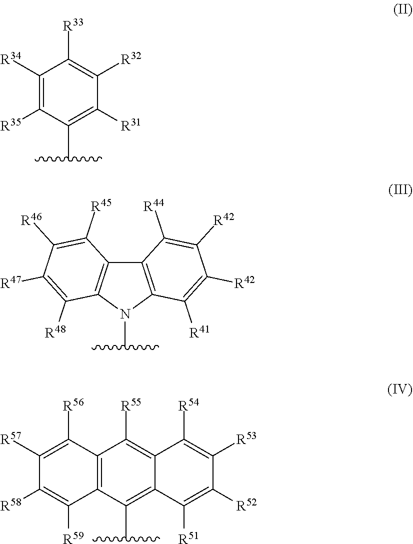

In formulas (II), (III), and (IV), each of R³¹⁻³⁵, R⁴¹⁻⁴⁸, or R⁵¹⁻⁵⁹ is independently chosen from (C₁-C₄₀)hydrocarbyl, (C₁-C₄₀)heterohydrocarbyl, —Si(R^{C})₃, —Ge(R^{C})₃, —P(R^{P})₂, —N(R^{N})₂, —N═CHR^{C}, —OR^{C}, —SR^{C}, —NO₂, —CN, —CF₃, R^{C}S(O)—, R^{C}S(O)₂—, (R^{C})₂C═N—, R^{C}C(O)O—, R^{C}OC(O)—, R^{C}C(O)N(R^{N})—, (R^{N})₂NC(O)—, halogen, or —H, provided at least one of R¹ or R⁸ is a radical having formula (II), formula (III), or formula (IV) where R^{C}, R^{N}, and R^{P} are as defined above.

In formula (I), each of R²⁻⁴, R⁵⁻⁷, and R⁹⁻¹⁶ is independently selected from (C₁-C₄₀)hydrocarbyl, (C₁-C₄₀)heterohydrocarbyl, —Si(R^{C})₃, —Ge(R^{C})₃, —P(R^{P})₂, —N(R^{N})₂, —N═CHR^{C}, —OR^{C}, —SR^{C}, —NO₂, —CN, —CF₃, R^{C}S(O)—, R^{C}S(O)₂—, (R^{C})₂C═N—, R^{C}C(O)O—, R^{C}OC(O)—, R^{C}C(O)N(R^{N})—, (R^{C})₂NC(O)—, halogen, and —H where R^{C}, R^{N}, and R^{P} are as defined above.

The catalyst system comprising a metal-ligand complex of formula (I) may be rendered catalytically active by any technique known in the art for activating metal-based catalysts of olefin polymerization reactions. For example, a metal-ligand complex of formula (I) may be rendered catalytically active by contacting the complex to, or combining the complex with, an activating co-catalyst. Suitable activating co-catalysts for use herein include alkyl aluminums; polymeric or oligomeric alumoxanes (also known as aluminoxanes); neutral Lewis acids; and non-polymeric, non-coordinating, ion-forming compounds (including the use of such compounds under oxidizing conditions). A suitable activating technique is bulk electrolysis. Combinations of one or more of the foregoing activating co-catalysts and techniques are also contemplated. The term “alkyl aluminum” means a monoalkyl aluminum dihydride or monoalkylaluminum dihalide, a dialkyl aluminum hydride or dialkyl aluminum halide, or a trialkylaluminum. Examples of polymeric or oligomeric alumoxanes include methylalumoxane, triisobutylaluminum-modified methylalumoxane, and isobutylalumoxane.

Lewis acid activators (co-catalysts) include Group 13 metal compounds containing from 1 to 3 (C₁-C₂₀)hydrocarbyl substituents as described herein. Examples of Group 13 metal compounds are tri((C₁-C₂₀)hydrocarbyl)-substituted-aluminum or tri((C₁-C₂₀)hydrocarbyl)-boron compounds; tri(hydrocarbyl)-substituted-aluminum, tri((C₁-C₂₀)hydrocarbyl)-boron compounds; tri((C₁-C₁₀)alkyl)aluminum, tri((C₆-C₁₈)aryl)boron compounds; and halogenated (including perhalogenated) derivatives thereof. In further examples, Group 13 metal compounds are tris(fluoro-substituted phenyl)boranes, tris(pentafluorophenyl)borane. An activating co-catalyst can be a tris((C₁-C₂₀)hydrocarbyl borate (e.g. trityl tetrafluoroborate) or a tri((C₁-C₂₀)hydrocarbyl) ammonium tetra((C₁-C₂₀)hydrocarbyl)borane (e.g. bis(octadecyl)methylammonium tetrakis(pentafluorophenyl)borane). As used herein, the term “ammonium” means a nitrogen cation that is a ((C₁-C₂₀)hydrocarbyl)₄N⁺, a ((C₁-C₂₀)hydrocarbyl)₃N(H)⁺, a ((C₁-C₂₀)hydrocarbyl)₂N(H)₂⁺, (C₁-C₂₀)hydrocarbylN(H)₃⁺, or N(H)₄⁺, wherein each (C₁-C₂₀)hydrocarbyl, when two or more are present, may be the same or different.

Combinations of neutral Lewis acid activators (co-catalysts) include mixtures comprising a combination of a tri((C₁-C₄)alkyl)aluminum and a halogenated tri((C₆-C₁₈)aryl)boron compound, especially a tris(pentafluorophenyl)borane; or combinations of such neutral Lewis acid mixtures with a polymeric or oligomeric alumoxane, and combinations of a single neutral Lewis acid, especially tris(pentafluorophenyl)borane with a polymeric or oligomeric alumoxane. Ratios of numbers of moles of (metal-ligand complex):(tris(pentafluoro-phenylborane):(alumoxane) [e.g., (Group 4 metal-ligand complex):(tris(pentafluoro-phenylborane):(alumoxane)] are from 1:1:1 to 1:10:30, or from 1:1:1.5 to 1:5:10.

The catalyst system comprising the metal-ligand complex of formula (I) can be activated to form an active catalyst composition by combination with one or more co-catalysts, for example, a cation forming co-catalyst, a strong Lewis acid, or combinations thereof. Suitable activating co-catalysts include polymeric or oligomeric aluminoxanes, especially methyl aluminoxane, as well as inert, compatible, noncoordinating, ion forming compounds. Exemplary suitable co-catalysts include, but are not limited to: modified methyl aluminoxane (MMAO), bis(hydrogenated tallow alkyl)methyl, tetrakis(pentafluorophenyl)borate(1⁻) amine, and combinations thereof.

One or more of the foregoing activating co-catalysts can be used in combination with each other. One preferred combination is a mixture of a tri((C₁-C₄)hydrocarbyl)aluminum, tri((C₁-C₄)hydrocarbyl)borane, or an ammonium borate with an oligomeric or polymeric alumoxane compound. The ratio of total number of moles of one or more metal-ligand complexes of formula (I) to total number of moles of one or more of the activating co-catalysts is from 1:10,000 to 100:1. The ratio can be at least 1:5000, or, at least 1:1000; and can be no more than 10:1 or no more than 1:1. When an alumoxane alone is used as the activating co-catalyst, preferably the number of moles of the alumoxane that are employed can be at least 100 times the number of moles of the metal-ligand complex of formula (I). When tris(pentafluorophenyl)borane alone is used as the activating co-catalyst, the ratio of the number of moles of the tris(pentafluorophenyl)borane that are employed to the total number of moles of one or more metal-ligand complexes of formula (I) can be from 0.5:1 to 10:1, from 1:1 to 6:1, or from 1:1 to 5:1. The remaining activating co-catalysts are generally employed in approximately mole quantities equal to the total mole quantities of one or more metal-ligand complexes of formula (I).

### Nonwoven and Reusable Outer Cover

A reusable outer cover formed from a nonwoven comprising the bicomponent fiber described above is disclosed herein. The nonwoven comprising bicomponent fibers can be formed via different techniques. Such techniques for forming the nonwoven disclosed herein include melt spinning, melt blown process, spunbond process, staple process, carded web process, air laid process, thermo-calendering process, adhesive bonding process, hot air bonding process, needle punch process, hydroentangling process, and electrospinning process. For example, in one embodiment, a reusable outer cover is formed from a spunbond nonwoven comprising the bicomponent fiber according to embodiments disclosed. In other embodiments, a reusable outer cover is formed from a meltblown nonwoven comprising the bicomponent fiber according to embodiments disclosed herein.

In embodiments, the nonwoven used to form the reusable outer cover is a coated nonwoven. For example, in embodiments, the nonwoven can receive a polyurethane dispersion coating. Without being bound by theory, such a coating can be applied to prevent leakage or transmission of liquid or insult when the reusable outer cover is worn.

In embodiments, the nonwoven used to form the reusable outer cover comprises at least 90 wt. % polyethylene, or at least 95 wt. % polyethylene, or at least 99 wt. % polyethylene, or at least 99.5 wt. % polyethylene, or at least 99.9 wt. % polyethylene, based on the overall weight of the reusable outer cover.

In embodiments, the reusable outer cover has a front region, a back region, and a crotch region disposed longitudinally between the front region and the back region, and a wearer-facing surface disposed opposite a garment-facing surface. In embodiments, the reusable outer cover (and the regions of the reusable outer cover) is a single layer of the nonwoven used to form the reusable outer layer. In other embodiments, the reusable outer layer is a dual layer, one layer of the nonwoven, and another layer of the same or different nonwoven according to embodiments disclosed herein.

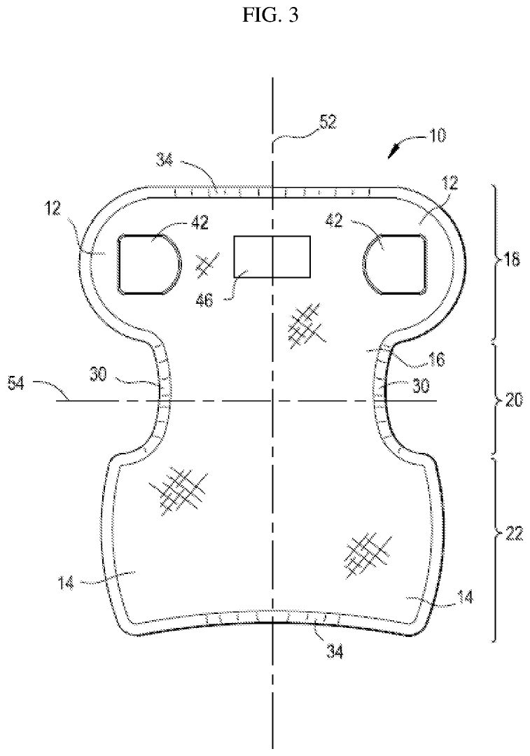

Referring now generally to FIG. 3, an example of a reusable outer cover 10 is shown. The reusable outer cover 10 is shown with a wearer-facing surface 16 up. Garment-facing surface is not shown in FIG. 3. The reusable outer cover 10 is a single layer nonwoven. The reusable outer cover 10 has longitudinal center line 52 and lateral centerline 54. The reusable outer cover 10 has front region 22, crotch region 20, and back region 18. When the reusable outer cover 10 is worn, front region 22 corresponds to wearer's front, crotch region 20 corresponds to the area between the wearer's legs, and back region 18 corresponds to wearer's back. The reusable outer cover 10 can have back side panels 12 and can have front side panels 14. The reusable cover 10 can have a pair of laterally opposed, longitudinally extending side edges 30, which can be curved. Each longitudinally extending side edge 30 can incorporate an elastic band to function as a cuff for the legs of the wearer. The reusable outer cover 10 can have a pair of longitudinally opposed, laterally extending side edges 34, and each longitudinally opposed, laterally extending side edge 34 can incorporate an elastic band to function as a waistband for the wearer. The reusable outer cover 10 can include an attachment point(s) 46 for attaching at least a portion of an absorbent article or component. For example, the attachment point(s) 46 can be a flap, pocket, or fastener(s) (e.g., Velcro®, snaps, or buttons) at the front region, back region, and/or crotch region for securing or attaching at least a portion of an absorbent article, such as an absorbent cloth, diaper, diaper core, insert, or nonwoven, that can absorb insult or liquid exudates, such as urine or feces. The reusable outer cover 10 can include fasteners 42 (e.g., buttons, snaps, or Velcro®) on either or both the back side panels 12 or front side panels 14 to attach the back side panels 12 to the front side panels 14 when the reusable outer cover 10 is worn by the wearer. For example, fasteners 42 on the back side panels 12 can connect to fasteners on the front side panels 14 on the garment-facing surface (not shown in FIG. 3).

In embodiments, the reusable outer cover has a basis weight of 50 to 150 gsm. All individual values of from 50 to 150 gsm are disclosed and included herein; for example, the reusable outer cover can have a basis weight as low as 50, 60, 70, 80, or 90 gsm and a basis weight as high as 150, 140, 130, 120, or 110 gsm.

In embodiments, the reusable outer cover is launderable. For example, in embodiments, the reusable outer cover can be laundered and can be reused after at least 5, at least 10, or at least 15 wash cycles.

The reusable outer cover according to embodiments disclosed herein is capable of retaining a significant portion of its extension force at 50% strain in the cross direction (CD). In embodiments, for example, the reusable outer cover after fifteen (15) wash cycles can have an extension force at 50% strain in the CD of at least 13.0 N at 110 gsm of nonwoven, at least 15.0 N at 110 gsm of nonwoven, at least 17.0 N at 110 gsm of nonwoven, at least 19.0 N at 110 gsm of nonwoven, or at least 21.0 N at 110 gsm of nonwoven, where extension force can be measured in accordance with the test method described below. The reusable outer cover can retain its extension force after being laundered, that is, the extension force of the reusable outer cover before being laundered and after being laundered is comparable. For example, in embodiments, the reusable outer cover retains at least 80%, at least 82%, at least 84%, at least 86%, or at least 88% of its extension force at 50% strain in the CD, after being laundered fifteen (15) times.

In embodiments, the reusable outer cover further comprises an attachment point on the wearer-facing surface of the reusable outer cover configured to attach to at least a portion of an absorbent article or component. For example, in embodiments, the attachment point can be a pocket that can fit at least a portion of an absorbent article (e.g., an absorbent core(s), layer(s), or insert(s) of a diaper).

In embodiments, the reusable outer cover formed from the nonwoven can have an optimal water vapor transmission rate (WVTR) and an extension force at 50% strain.

In embodiments, the reusable outer cover has a WVTR of at least 8,000 g/m²/24 hr at 110 gsm of the nonwoven, where WVTR can be measured in accordance with the test method described below. All individual values and subranges of at least 8,000 g/m²/24 hr are disclosed and included herein; for example, the reusable outer cover can have a WVTR of at least 8,000 g/m²/24 hr, at least 10,000 g/m²/24 hr, at least 12,000 g/m²/24 hr, at least 16,000 g/m²/24 hr, at least 20,000 g/m²/24 hr, at least 24,000 g/m²/24 hr, or at least 28,000 g/m²/24 hr, and can have an upper limit of 40,000 g/m²/24 hr, where WVTR can be measured in accordance with the test method described below.

In embodiments, the reusable outer cover has an extension force at 50% strain in the cross direction (CD) of greater than 13.0 N at 110 gsm of nonwoven, where extension force at 50% strain can be measured in accordance with the test method described below. All individual values and subranges of an extension force at 50% strain in the CD of greater than 13.0 N are disclosed and included herein; for example, the reusable outer cover can have an extension force at 50% strain in the CD of greater than 13.0 N, greater than 17.0 N, greater than 21.0 N, greater than 25.0 N, and can have an upper limit of 40.0 N at 110 gsm of nonwoven, where extension force at 50% strain in the CD can be measured in accordance with the test method described below.

### Test Methods

### Density

Density is measured in accordance with ASTM D-792, and expressed in grams/cm³ (g/cm³).

### Melt Index (I2)

Melt Index is measured in accordance with ASTM D 1238 at 190° Celsius and 2.16 kg, and is expressed in grams eluted/10 minutes (g/10 min).

Differential Scanning calorimetry (DSC)

Differential scanning calorimetry is used to measure highest peak melting temperature (Tm) and highest crystallization temperature (Tc) according to ASTM D3895-14. Approximately 0.5 gram of sample is compression molded into a film at 25,000 psi and 190° Celsius for 10-15 seconds. A 5 to 8 mg sample is weighed and placed in a DSC aluminum pan with the lid crimped on the pan to ensure a closed atmosphere. For cooling and second heat information, the sample is heated at a rate of 10° Celsius/minute to 150° Celsius and held isothermally for 5 minutes. The sample is then cooled at a rate of 10° Celsius/minute to −40° Celsius, held isothermally for 5 minutes before being heated at a rate of 10° Celsius/minute to 150° Celsius.

Conventional Gel Permeation Chromatography (conventional GPC)

The chromatographic system consists of a PolymerChar GPC-IR (Valencia, Spain) high temperature GPC chromatograph equipped with an internal IR5 infra-red detector (IR5). The autosampler oven compartment is set at 160° C. and the column compartment is set at 150° C. The columns used are 4 Agilent “Mixed A” 30 cm 20-micron linear mixed-bed columns. The chromatographic solvent used is 1,2,4 trichlorobenzene and contains 200 ppm of butylated hydroxytoluene (BHT). The solvent source is nitrogen sparged. The injection volume used is 200 microliters and the flow rate is 1.0 milliliter/minute.

Calibration of the GPC column set is performed with at least 20 narrow molecular weight distribution polystyrene standards with molecular weights ranging from 580 to 8,400,000 g/mol and are arranged in 6 “cocktail” mixtures with at least a decade of separation between individual molecular weights. The standards are purchased from Agilent Technologies. The polystyrene standards are prepared at 0.025 grams in 50 milliliters of solvent for molecular weights equal to or greater than 1,000,000 g/mol, and 0.05 grams in 50 milliliters of solvent for molecular weights less than 1,000,000 g/mol. The polystyrene standards are dissolved at 80° C. with gentle agitation for 30 minutes. The polystyrene standard peak molecular weights are converted to ethylene/alpha-olefin interpolymer molecular weights using the following equation (as described in Williams and Ward, J. Polym. Sci., Polym. Let., 6, 621 (1968)):

M_{polyethylene}=A×(M_{polystyrene})^{B} (Eq. 1)

where M is the molecular weight, A has a value of 0.4315 and B is equal to 1.0.

A fifth order polynomial is used to fit the respective ethylene/alpha-olefin interpolymer-equivalent calibration points. A small adjustment to A (from approximately 0.39 to 0.44) is made to correct for column resolution and band-broadening effects such that NIST standard NBS 1475 is obtained at a molecular weight of 52,000 g/mol.

The total plate count of the GPC column set is performed with Eicosane (prepared at 0.04 g in 50 milliliters of TCB and dissolved for 20 minutes with gentle agitation). The plate count (Equation 2) and symmetry (Equation 3) are measured on a 200 microliter injection according to the following equations:

$$Plate Count = 5.54 \times {( \frac{R {V}_{Peak Max}}{Peak Width at half height} )}^{2} \qquad ( Eq . 2 )$$

where RV is the retention volume in milliliters, the peak width is in milliliters, the Peak Max is the maximum height of the peak, and half height is one half of the height of the peak maximum.

$$Symmetry = \frac{( Rear Peak R {V}_{one tenth height} - R {V}_{Peak max} )}{( R {V}_{Peak max} - Front Peak R {V}_{one tenth height} )} \qquad ( Eq . 3 )$$

where RV is the retention volume in milliliters and the peak width is in milliliters, Peak max is the maximum position of the peak, one tenth height is one tenth of the height of the peak maximum, and where rear peak refers to the peak tail at later retention volumes than the Peak max and where front peak refers to the peak front at earlier retention volumes than the Peak max. The plate count for the chromatographic system should be greater than 22,000 and symmetry should be between 0.98 and 1.22.

Samples are prepared in a semi-automatic manner with the PolymerChar “Instrument Control” Software, wherein the samples are weight-targeted at 2 mg/ml, and the solvent (contained 200 ppm BHT) is added to a pre nitrogen-sparged septa-capped vial, via the PolymerChar high temperature autosampler. The samples are dissolved for 3 hours at 160° C. under “low speed” shaking.

The calculations of M_{n(GPC)}, M_{w(GPC)}, and M_{z(GPC)} are based on GPC results using the internal IR5 detector (measurement channel) of the PolymerChar GPC-IR chromatograph according to Equations 5a-c, using PolymerChar GPCOne™ software, the baseline-subtracted IR chromatogram at each equally-spaced data collection point i (IRᵢ) and the ethylene/alpha-olefin interpolymer equivalent molecular weight obtained from the narrow standard calibration curve for the point i (M_{polyethylene,i} in g/mol) from Equation 1. Subsequently, a GPC molecular weight distribution (GPC-MWD) plot (wt_{GPC}(lg MW) vs. lg MW plot, where wt_{GPC}(lg MW) is the weight fraction of the interpolymer molecules with a molecular weight of lg MW) can be obtained. Molecular weight is in g/mol and wt_{GPC}(lg MW) follows the Equation 4.

∫wt_{GPC}(lg MW)d lg MW=1.00 (Eq. 4)

Number-average molecular weight M_{n(GPC)}, weight-average molecular weight M_{w(GPC)} and z-average molecular weight M_{z(GPC)} can be calculated as the following equations.

$${Mn}_{( GPC )} = \frac{{\sum }^{i} {IR}_{i}}{{\sum }^{i} ( {IR}_{i} / {M}_{polyethylene {,}_{i}} )} \qquad ( Eq . 5 a )$$

$${Mw}_{( GPC )} = \frac{{\sum }^{i} ( {IR}_{i} ⋆ {M}_{polyethylene {,}_{i}} )}{{\sum }^{i} {IR}_{i}} \qquad ( Eq . 5 b )$$

$${Mz}_{( GPC )} = \frac{{\sum }^{i} ( {IR}_{i} ⋆ {M}_{{polyethylene}_{, i}}^{2} )}{{\sum }^{i} ( {IR}_{i} ⋆ {M}_{polyethylene {,}_{i}} )} \qquad ( Eq . 5 c )$$

In order to monitor the deviations over time, a flow rate marker (decane) is introduced into each sample via a micropump controlled with the PolymerChar GPC-IR system. This flow rate marker (FM) is used to linearly correct the pump flow rate (Flowrate(nominal)) for each sample by RV alignment of the respective decane peak within the sample (RV(FM Sample)) to that of the decane peak within the narrow standards calibration (RV(FM Calibrated)). Any changes in the time of the decane marker peak are then assumed to be related to a linear-shift in flow rate (Flowrate(effective)) for the entire run. To facilitate the highest accuracy of a RV measurement of the flow marker peak, a least-squares fitting routine is used to fit the peak of the flow marker concentration chromatogram to a quadratic equation. The first derivative of the quadratic equation is then used to solve for the true peak position. After calibrating the system based on a flow marker peak, the effective flow rate (with respect to the narrow standards calibration) is calculated as Equation 6. Processing of the flow marker peak is done via the PolymerChar GPCOne™ Software. Acceptable flow rate correction is such that the effective flowrate should be within 0.5% of the nominal flowrate.

Flow rate_{effective}=Flow rateₙₒₘᵢₙₐₗ×(RV(FM_{calibrated})/RV(FM_{Sample})) (Eq. 6)

### Extension Force

The Outer Cover Composite Extension Forces are measured on a constant rate of extension tensile tester with computer interface (a suitable instrument is the MTS Alliance using Testworks 4.0 Software, as available from MTS Systems Corp., Eden Prairie, Minn.) using a load cell for which the forces measured are within 10% to 90% of the limit of the cell. Both the movable (upper) and stationary (lower) pneumatic jaws are fitted with rubber faced grips wider than the width of the specimen. The gage length is 25.4 mm and the data acquisition rate is 100 Hz.

Program the tensile tester to extend the specimen to 110% strain at a crosshead speed of 254 mm/min. and then return to the original crosshead position. Program the software to report the force (N) at 50% strain and 100% strain.

Using a JDC precision cutter (Thwing Albert) cut along the longitudinal axis of the outer cover a 1″ wide strip in the longitudinal direction of the outer cover that is 3″ long. If there are multiple layers of the outer cover the specimen should be cut though all layers. The composite should be tested as a whole and also as the individual layers. Any single specimen (either composite or single layer) should only be tested once.

Set the gage length to 25.4 mm, zero the crosshead and zero the load cell. Insert the specimen into the upper grips, aligning it vertically within the upper and lower jaws and close the upper grips. Insert the specimen into the lower grips and close. The specimen is placed under enough tension to eliminate any slack, but less than 0.05N of force on the load cell. Start the tensile tester's program and collect data. Report the force (N) at 50% extension for the composite (multi-layered) specimen to ±0.01 N. Report the force (N) at 100% extension for each of the single layered specimens to ±0.01 N.

### Water Vapor Transmission Rate (WVTR)

Water Vapor Transmission rate (WVTR) is measured according to EDANA/INDA Worldwide Strategic Partners Method WSP 70.4 (08) using a Permatran-W model 100K (MOCON, Minnesota, Minn.). The test method is ran as per the WSP standard test, using a test apparatus temperature of 37.8 C, a nitrogen flowrate of 120 SCCM, and the standard mode with 2 cycles and 5 minute exam time. Each cell is individually adjusted to a relative humidity (RH) of 60%±1.5%. The standard reference film (S/N 1008WK089 from MOCON) is ran prior to testing the samples in order to ensure that the equipment is running properly. The standard reference film results are within ±10% of the values reported by MOCON.

Using scissors or a die cut a specimen 35 mm in diameter. The side of the outer cover which normally faces the skin is oriented toward the water for testing. Report the WVTR as g/m²/24 hr to the nearest 1 g/m²/24 hr.

### Examples

### Production of Ethylene/Alpha-Olefin Interpolymers

Polymer 1 (Poly. 1) and Polymer 2 (Poly. 2), which are ethylene/alpha-olefin interpolymers, are prepared according to the following process and tables.

All raw materials (monomer and comonomer) and the process solvent (a narrow boiling range high-purity isoparaffinic solvent, Isopar-E) are purified with molecular sieves before introduction into the reaction environment. Hydrogen is supplied pressurized as a high purity grade and is not further purified. The reactor monomer feed stream is pressurized via a mechanical compressor to above reaction pressure. The solvent and comonomer (if present) feed is pressurized via a pump to above reaction pressure. The individual catalyst components are manually batch diluted with purified solvent and pressured to above reaction pressure. All reaction feed flows are measured with mass flow meters and independently controlled with computer automated valve control systems.

Reactor configuration is either single reactor operation or dual series reactor operation as specified in Table 2.

Either a single reactor system or a two reactor system in a series configuration is used. Each reactor is a continuous solution polymerization reactor consisting of a liquid full, non-adiabatic, isothermal, circulating, loop reactor which mimics a continuously stirred tank reactor (CSTR) with heat removal. Independent control of all fresh solvent, monomer, comonomer (if present), hydrogen, and catalyst component feeds is possible. The total fresh feed stream to each reactor (solvent, monomer, comonomer [if present], and hydrogen) is temperature controlled typically between 15-50° C. to maintain a single solution phase by passing the feed stream through a heat exchanger. The total fresh feed to each polymerization reactor is injected into the reactor at two locations with approximately equal reactor volumes between each injection location. The fresh feed is controlled with each injector receiving half of the total fresh feed mass flow. The catalyst components are injected into the polymerization reactor through injection nozzles to introduce the components into the center of the reactor flow. The primary catalyst component feed is computer controlled to maintain the reactor monomer conversion at the specified values. The cocatalyst component(s) is/are fed based on calculated specified molar ratios to the primary catalyst component Immediately following each reactor feed injection location, the feed streams are mixed with the circulating polymerization reactor contents with static mixing elements. The contents of each reactor are continuously circulated through heat exchangers responsible for removing much of the heat of reaction and with the temperature of the coolant side responsible for maintaining an isothermal reaction environment at the specified temperature. Circulation around each reactor loop is provided by a pump.

In dual series reactor configuration the effluent from the first polymerization reactor (containing solvent, monomer, comonomer [if present], hydrogen, catalyst components, and polymer) exits the first reactor loop and is added to the second reactor loop. In all reactor configurations the final reactor effluent (second reactor effluent for dual series or the single reactor effluent) enters a zone where it is deactivated with the addition of and reaction with a suitable reagent (water). At this same reactor exit location other additives are added for polymer stabilization (e.g., antioxidants suitable for stabilization during extrusion and fabrication include Octadecyl 3,5-Di-Tert-Butyl-4-Hydroxyhydrocinnamate, Tetrakis(Methylene(3,5-Di-Tert-Butyl-4-Hydroxyhydrocinnamate))Methane, and Tris(2,4-Di-Tert-Butyl-Phenyl) Phosphite).

Following catalyst deactivation and additive addition, the reactor effluent enters a devolatization system where the polymer is removed from the non-polymer stream. The isolated polymer melt is pelletized and collected. The non-polymer stream passes through various pieces of equipment which separate most of the ethylene which is removed from the system. Most of the solvent and unreacted comonomer (if present) is recycled back to the reactor after passing through a purification system. A small amount of solvent and comonomer (if present) is purged from the process.

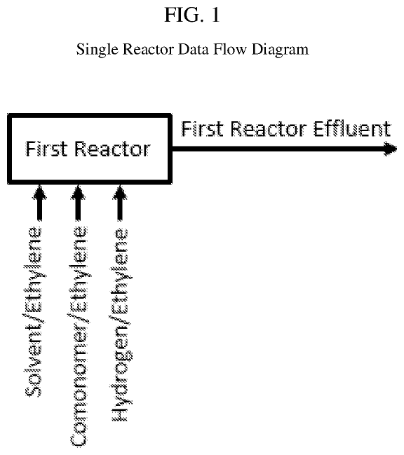

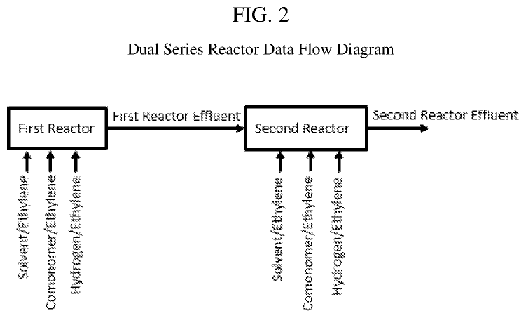

The reactor stream feed data flows that correspond to the values in Table 2 used to produce the polymers are graphically described in FIG. 1 and FIG. 2. The data are presented such that the complexity of the solvent recycle system is accounted for and the reaction system can be treated more simply as a once through flow diagram.

TABLE 1
Catalyst Components

| | |
|---|---|
| Primary Catalyst Component 1 | 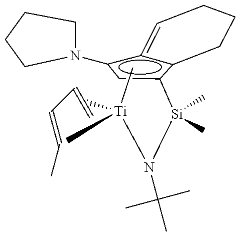 |
| Primary Catalyst Component 2 | 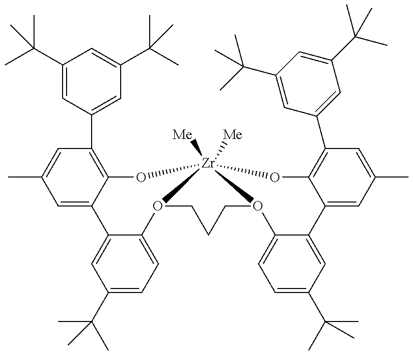 |
| Primary Catalyst Component 3 | 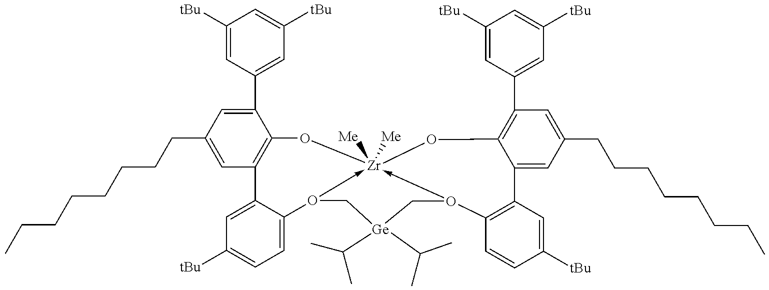 |
| Primary Catalyst Component 4 | 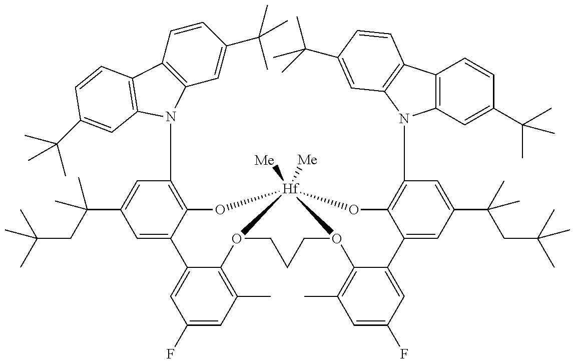 |
| Primary Catalyst | A Ziegler-Natta type catalyst. The heterogeneous Ziegler-Natta type |
| Component 5 | catalyst-premix was prepared substantially according to U.S. Pat. No. |
| | 4,612,300, by sequentially adding to a volume of ISOPAR E, a slurry |
| | of anhydrous magnesium chloride in ISOPAR E, a solution of EtAlCl₂ |
| | in heptane, and a solution of Ti(O—iPr)₄ in heptane, to yield a |
| | composition containing a magnesium concentration of 0.20 M and a |
| | ratio of Mg/Al/Ti of 40/12.5/3. An aliquot of this composition was |
| | further diluted with ISOPARE-E to yield a final concentration of 500 |
| | ppm Ti in the slurry. An aliquot of this composition can be further |
| | diluted with ISOPAR-E if required. The catalyst premix was contacted |
| | with a dilute solution of Et₃Al, in the molar Al to Ti ratio specified in |
| | Table 2, to give the active catalyst. |
| Primary Catalyst Component 6 | 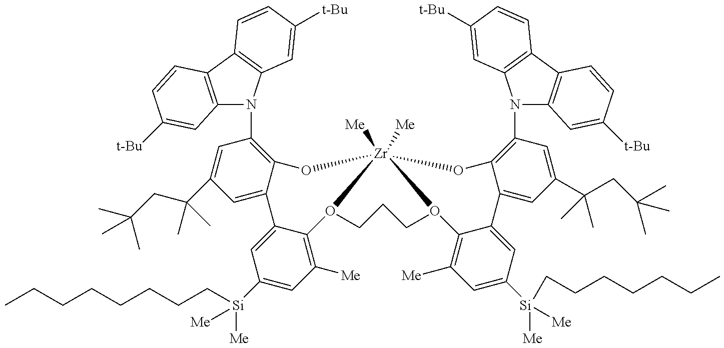 |
| Co-catalyst A | bis(hydrogenated tallow alkyl)methylammonium tetrakis(pentafluoro- |
| | phenyl)borate(1−) |
| Co-catalyst B | Modified methyl aluminoxane |
| Co-catalyst C | Triethyl aluminum |

TABLE 2A
Production Configurations for Poly. 1

| | | Poly. 1 |
|---|---|---|
| Reactor Configuration | Type | Dual Series |
| Comonomer type | Type | 1-octene |
| First Reactor Feed Solvent/ | g/g | 3.13 |
| Ethylene Mass Flow Ratio | | |
| First Reactor Feed Comonomer/ | g/g | 0.331 |
| Ethylene Mass Flow Ratio | | |
| First Reactor Feed Hydrogen/ | g/g | 3.35E−04 |
| Ethylene Mass Flow Ratio | | |
| First Reactor Temperature | ° C. | 160 |
| First Reactor Pressure | barg | 40 |
| First Reactor Ethylene | % | 89.7 |
| Conversion | | |
| First Reactor Catalyst Type | Type | Primary Catalyst |
| (See also Table 1) | | component 3 |
| First Reactor Co-Catalyst 1 Type | Type | Co-catalyst A |
| (See also Table 1) | | |
| First Reactor Co-Catalyst 2 Type | Type | Co-catalyst B |
| (See also Table 1) | | |
| First Reactor Co-Catalyst 1 to | mol/mol | 1.2 |
| Catalyst Molar Ratio (B to | | |
| Catalyst Metal ratio) | | |
| First Reactor Co-Catalyst 2 to | mol/mol | 39 |
| Catalyst Molar Ratio (Al to | | |
| Catalyst Metal ratio) | | |
| First Reactor Residence Time | min | 17.8 |
| Percentage of Total Ethylene | wt % | 37.9% |
| Feed to First Reactor | | |
| Second Reactor Feed Solvent/ | g/g | 2.45 |
| Ethylene Mass Flow Ratio | | |
| Second Reactor Feed | g/g | 0.134 |
| Comonomer/Ethylene Mass | | |
| Flow Ratio | | |
| Second Reactor Feed Hydrogen/ | g/g | 6.27E−04 |
| Ethylene Mass Flow Ratio | | |
| Second Reactor Temperature | ° C. | 195 |
| Second Reactor Pressure | barg | 40 |
| Second Reactor Ethylene | % | 91.6 |
| Conversion | | |
| Second Reactor Catalyst Type | Type | Primary Catalyst |
| (See also Table 1) | | component 6 |
| Second Reactor Co-Catalyst 1 | Type | Co-catalyst A |
| Type | | |
| (See also Table 1) | | |
| Second Reactor Co-Catalyst 2 | Type | Co-catalyst B |
| Type | | |
| (See also Table 1) | | |
| Second Reactor Co-Catalyst 1 to | mol/mol | 5.5 |
| Catalyst Molar Ratio (B to | | |
| Catalyst Metal ratio) | | |
| Second Reactor Co-Catalyst 2 to | mol/mol | 2249 |
| Catalyst Molar Ratio (Al to | | |
| Catalyst Metal ratio) | | |
| Second Reactor Residence Time | min | 7.5 |

TABLE 2B
Production Configurations for Poly. 2

| | | Poly. 2 |
|---|---|---|
| Reactor Configuration | Type | Single |
| Comonomer type | Type | 1-hexene |
| Reactor Feed Solvent/ | g/g | 3.47 |
| Ethylene Mass Flow Ratio | | |
| Reactor Feed Comonomer/ | g/g | 0.009 |
| Ethylene Mass Flow Ratio | | |
| Reactor Feed Hydrogen/ | g/g | 3.10E−04 |
| Ethylene Mass Flow Ratio | | |
| Reactor Temperature | ° C. | 185 |
| Reactor Pressure | barg | 38 |
| Reactor Ethylene | % | 93.6 |
| Conversion | | |
| Reactor Catalyst Type | Type | Primary Catalyst |
| (See also Table 1) | | component 2 |
| Reactor Co-Catalyst 1 Type | Type | Co-catalyst A |
| (See also Table 1) | | |
| Reactor Co-Catalyst 2 Type | Type | Co-catalyst C |
| (See also Table 1) | | |
| Reactor Co-Catalyst 1 to | mol/mol | 1.1 |
| Catalyst Molar Ratio (B to | | |
| Catalyst Metal ratio) | | |
| Reactor Co-Catalyst 2 to | mol/mol | 2 |
| Catalyst Molar Ratio (Al to | | |
| Catalyst Metal ratio) | | |
| Reactor Residence Time | min | 12.6 |

Table 3 below provides the Melt Index (I2), Density, Mw_{(GPC)}/Mn_{(GPC)}, and highest crystallization temperature (Tc), of Poly. 1 and Poly 2.

TABLE 3
Characteristics of Poly. 1 and Poly. 2

| | | | | Highest |
|---|---|---|---|---|
| | Melt Index | Density | | Crystallization |
| | (g/10 min) | (g/cm³) | Mw_{(GPC)}/Mn_{(GPC)} | Temp. (Tc) |
| Poly. 1 | 19 | 0.935 | 2.1 | 113° C. |
| Poly 2. | 19 | 0.949 | 2.2 | 114° C. |

### Formation of Fibers and Nonwovens

A spunbond nonwoven is formed from the bicomponent fibers and produced on a single beam Reicofil 4 spunbond line in a 50:50 (in weight percent) concentric core:sheath configuration. The sheath of the bicomponent fiber is Poly. 1 (described above), and the core of the bicomponent fiber is Poly 2 (described above). The machine (Reicofil 4 spunbond line) is equipped with a spinneret having 7022 holes (6861 holes/m) and an exit diameter of each hole of 0.6 mm. The hole has a L/D ratio of 4. The polymer melt temperature is set at approximately 230° C. Fibers are collected at the maximum sustainable cabin air pressure while maintaining stable fiber spinning and transformed into a nonwoven. Bonding of the web takes place between an engraved roll and a smooth roll with a nip pressure of 70 daN/cm. The oil temperature of the engraved roll is adjusted for achieving the best bonding without overwrapping the nonwoven onto the roll. The oil temperature of the smooth roll is kept at 2° C. lower than that of the engraved roll. The cabin pressure is 5400 Pa, filament speed is 4582, throughput rate is 0.56 gram/hole/minute, and optimized engraved roll temperature is 124° C. Spunbond nonwovens having a basis weight of 110 gsm are formed for use to form a reusable outer cover.

### Formation of the Reusable Outer Cover

A reusable outer cover is constructed from the spunbond nonwoven having a basis weight of 110 gsm and is designated as Inventive Example 1. Inventive Example 1 has a construction that corresponds in aspects to the illustration of the reusable outer cover depicted in FIG. 3. Inventive Example 1 is formed from cutting the spunbond nonwoven such that it has a front region, a back region, and a crotch region disposed longitudinally between the front region and the back region, and a wearer-facing surface disposed opposite a garment-facing surface, and has front side panels and back side panels. Inventive Example 1 is cut so that it has a pair of curved, laterally opposed, longitudinally extending side edges, and each longitudinally extending side edge has an elastic band sewn into it so as to function as a cuff for the legs of the wearer. Inventive Example 1 is cut so that it has a pair of longitudinally opposed, laterally extending side edges and each longitudinally opposed, laterally extending side edge has an elastic band sewn into it to function as a waistband for the wearer. Inventive Example 1 includes a flap at the front region for securing or attaching at least a portion of an absorbent article, and includes Velcro® on the back side panels as fasteners to attach the back side panels to the front side panels when worn by the wearer.

Inventive Example 1 is a reusable outer cover that is formed from a nonwoven comprising all polyethylene (specifically, two ethylene/alpha-olefin interpolymers as described above), and so is designed for full compatibility in polyethylene recycling streams. Inventive Example 1 is a single layer nonwoven.

Comparative Example 1 is a Thirsties Baby Duo Wrap Diaper Cover™, size 2, available from Thirsties, Inc., Loveland, Colo.

The examples are tested for WVTR and extension force at 50% strain in the cross direction (CD) and in the machine direction (MD). The results are reported in Table 4. As shown in Table 4, Inventive Example 1 has a significantly and surprisingly higher WVTR and extension force at 50% in the CD. The examples are laundered and subjected to 15 wash cycles (5 in cold water, 5 in warm water, and 5 in hot water) and then tested again for extension force at 50% strain in the CD. The retention of extension force is calculated. The results are reported in Table 5. As shown, Inventive Example 1 has a higher retention of extension force after being laundered than the Comparative Example 1.

TABLE 4
WVTR and Extension Force at 50% Strain

| | | Extension Force at | Extension Force at |
|---|---|---|---|
| | | 50% Strain in the | 50% Strain in the |
| | WVTR (g/m²/24 hr) | CD (N) | MD (N) |
| Inventive Example 1 | 29,293 g/m²/24 hr | 26.8N | 67.1N |
| Comparative | 552 g/m²/24 hr | 11.8N | 65.9N |
| Example 1 | | | |

TABLE 5
Extension Force at 50% Strain After 15 Wash Cycles

| | Extension Force | Extension Force at | Retention of | Retention of |
|---|---|---|---|---|
| | at 50% Strain in | 50% Strain in the | Extension Force in | Extension Force |
| | the CD After 15 | MD After 15 Wash | CD After 15 Wash | in MD After 15 |
| | Wash Cycles (N) | Cycles (N) | Cycles (%) | Wash Cycles (%) |
| Inventive | 22.9N | 59.2N | 85% | 88% |
| Example 1 | | | | |
| Comparative | 9.5N | 49.2N | 81% | 75% |
| Example 1 | | | | |

Every document cited herein, if any, including any cross-referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A reusable outer cover suitable for use as a cover to an absorbent article, the reusable outer cover having a front region, a back region, and a crotch region disposed longitudinally between the front region and the back region, and a wearer-facing surface disposed opposite a garment-facing surface, the reusable outer cover formed from a nonwoven comprising:
a bicomponent fiber comprising a first region and a second region;
the first region comprising a first polymer; and
the second region comprising a second polymer.

2. The reusable outer cover of claim 1, wherein the first polymer is a first ethylene/alpha-olefin interpolymer.

3. The reusable outer cover of claim 1, wherein the second polymer is a second ethylene/alpha-olefin interpolymer.

4. The reusable outer cover of claim 1, wherein the bicomponent fiber is arranged in a core-sheath configuration, wherein the first region is a core region and the second region is a sheath region, and the sheath region surrounds the core region.

5. The reusable outer cover of claim 1, wherein the nonwoven is a coated nonwoven.

6. The reusable outer cover of claim 1, wherein the reusable outer cover has at least one of the following properties: an extension force at 50% strain in the cross direction of greater than 13.0 N at 110 gsm of the nonwoven; and a WVTR of at least 8,000 g/m²/24 hr at 110 gsm of the nonwoven.

7. The reusable outer cover of claim 1, wherein the reusable outer cover further comprises an attachment point on the wearer-facing surface of the reusable outer cover configured to attach to at least a portion of an absorbent article.

8. The reusable outer cover of claim 1, wherein the reusable outer cover has a basis weight of 50 to 150 gsm.

9. The reusable outer cover of claim 1, wherein the reusable outer cover has an extensional force at 50% in the cross direction after fifteen wash cycles of greater than 10.0 N at 110 gsm of the nonwoven.

10. The reusable outer cover of claim 1, wherein the reusable outer cover has an extensional force at 50% in the machine direction after fifteen wash cycles of greater than 52.0 N at 110 gsm of the nonwoven.
